(19) United States
(12) Patent Application Publication
(10) Pub. No.: US 2021/0030593 A1
(43) Pub. Date: Feb. 4, 2021

(54) SYSTEM FOR PREVENTING DAMAGE TO COSMETIC EYELASHES

(71) Applicant: Lea Elizabeth Kellogg, Fort Lauderdale, FL (US)

(72) Inventor: Lea Elizabeth Kellogg, Fort Lauderdale, FL (US)

(21) Appl. No.: 16/942,163

(22) Filed: Jul. 29, 2020

Related U.S. Application Data
(60) Provisional application No. 62/881,047, filed on Jul. 31, 2019.

(51) Int. Cl. A61F 9/02; G02C 3/00

(52) U.S. Cl. CPC A61F 9/026; A63B 33/002; G02C 3/003

(57) ABSTRACT

A cosmetic lash protection device comprising a pair of eye members. Each eye member including a rim defining an interior volume configured to allow the cosmetic lashes to move freely therein. An adhesive disposed on each rim, wherein the adhesive is configured to removably attach the system to a user's face. Systems and methods are provided.

### CROSS-REFERENCE TO RELATED APPLICATIONS

This application claims priority to U.S. provisional patent application Ser. No. 62/881,047 filed on Jul. 31, 2019 and is hereby incorporated by refences in its entirety.

### TECHNICAL FIELD

The present invention relates to the field of cosmetic accessories, and more specifically to the field of eyelash protection devices.

### BACKGROUND

The idea of lengthening eyelashes as a way of enhancing looks is not a new one. While not yet having developed a technique to do so at the time, many books and publications on fashion in the late 1800s advised on how to lengthen eyelashes. Methods advised at the time included cutting the ends off of eyelashes to encourage longer growth. Other treatment methods said to enhance growth included the use of pomade and washing eyelashes with a mixture of walnut leaves and water. In 1882, it was reported in the press that residents of Paris had started sewing hair into their eyelids to create fake eyelashes. This painful precursor to modern eyelash extensions was also reported in 1899 in the Scottish press.

In 1902, famous hair stylist and inventor Karl Nessler patented a method in the United Kingdom to weave artificial eyelashes, and by 1911, false eyelashes were patented in the United States. However, it was popular culture that lead to the widespread use of false eyelash extensions. In 1916, Hollywood film director, D. W. Griffith, wanted one of his actresses to wear exaggerated eyelashes. He asked a manufacturer of natural hair wigs to make human eyelashes. Thereafter, as techniques improved, false eyelashes became increasingly popular with the general public in the 1930s and again in the 1960s.

Today, cosmetic eyelashes are readily available and are typically used for important events, special occasions, nighttime party makeovers and in the world of entertainment and fashion. Cosmetic eyelash extensions are used to enhance the length, curliness, fullness, and thickness of natural eyelashes. The extensions may be made from several materials including mink, synthetic, or horsehair and come as individuals, clusters, and most commonly, lash strips.

In the United States, eyelash extension services can range from $100 to $500, depending on the number and type of lashes being used, the skill level of the cosmetician or, the venue where the extensions are applied. Moreover, because an average person might have anywhere from one hundred to two hundred lashes per eye, it can take from one to three hours to attach a full, new set. Cosmetic eyelash extensions are applied using an adhesive glue, and are not designed to be worn when showering, sleeping or swimming. Consequently, eyelash extensions usually last between 3-4 weeks or require a refill every 2-3 weeks or a partial set every four weeks.

Therefore, there is a need in the art for a system that is capable of extending the lifespan of cosmetic eyelashes, and more particularly, for preventing damage to cosmetic eyelashes.

### SUMMARY

In one embodiment, a cosmetic lash protection device is provided. The device includes a pair of eye members. Each eye member includes a rim defining an interior volume and is configured to allow the cosmetic lashes to move freely therein. An adhesive is disposed on each rim. The adhesive is configured to removably attach the device to a user's face. Systems and methods are provided.

In one embodiment, the cosmetic lash protection device comprises a pair of eye members. Each eye member includes a rim and an interior volume configured to allow the cosmetic lashes to move freely therein. A connector member is provided. An attachment mechanism is configured for connecting the eye members with the connector member. -A replaceable adhesive disposed on each rim, wherein the adhesive is configured to removably attach the system to a user's face.

In one embodiment, a cosmetic lash protection system is provided. The system includes a user wearing a pair of cosmetic lashes. A pair of eye members configured for placement over the eyes of the user. Each eye member includes a rim defining an interior volume configured to allow the cosmetic lashes to move freely therein. A connector member connects the eye members to each other. An adhesive disposed on each rim, wherein the adhesive is configured to removably attach the system to a user's face.

A system for preventing damage to cosmetic eyelashes is disclosed. This Summary is provided to introduce a selection of disclosed concepts in a simplified form that are further described below in the Detailed Description including the drawings provided. This Summary is not intended to identity key features or essential features of the claimed subject matter. Nor is this Summary intended to be used to limit the claimed subject matter's scope.

In one embodiment, a system for preventing damage to cosmetic eyelashes is disclosed. The system includes a pair of eye members. A connector member connects the eye members to each other. Each eye member has a transparent portion and a rim on a rear edge of each eye member. An adhesive is disposed on each rim and is configured to removably attach the system to a user's face.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 is a front view of a system for preventing damage to cosmetic eyelashes, according to an example embodiment of the present invention;

FIG. 2 is a top perspective view of the system according to an example embodiment of the present invention;

FIG. 3 is a top view of the system according to an example embodiment of the present invention;

FIG. 4 is a back perspective view of the system according to an example embodiment of the present invention;

FIG. 5 is a back view of the system according to an example embodiment of the present invention

FIG. 6 is a side perspective view of the system according to an example embodiment of the present invention;

FIG. 7 is a side view of the system according to an example embodiment of the present invention;

FIG. 8 is a side perspective view of the system according to an example embodiment of the present invention;

### DETAILED DESCRIPTION

The following detailed description refers to the accompany mg drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims.

The present invention satisfies a need in the art by providing a system that is capable of preventing damage and extending the lifespan of cosmetic eyelashes. The system includes a pair of eye members having an interior volume that are configured to allow cosmetic lashes to move freely within the interior volume. Additionally, a water-resistant adhesive is disposed on each rim of the eye member such that the system remains on the user's face when worn by the user while bathing or other activities without using a strap to attach the system to the user's face.

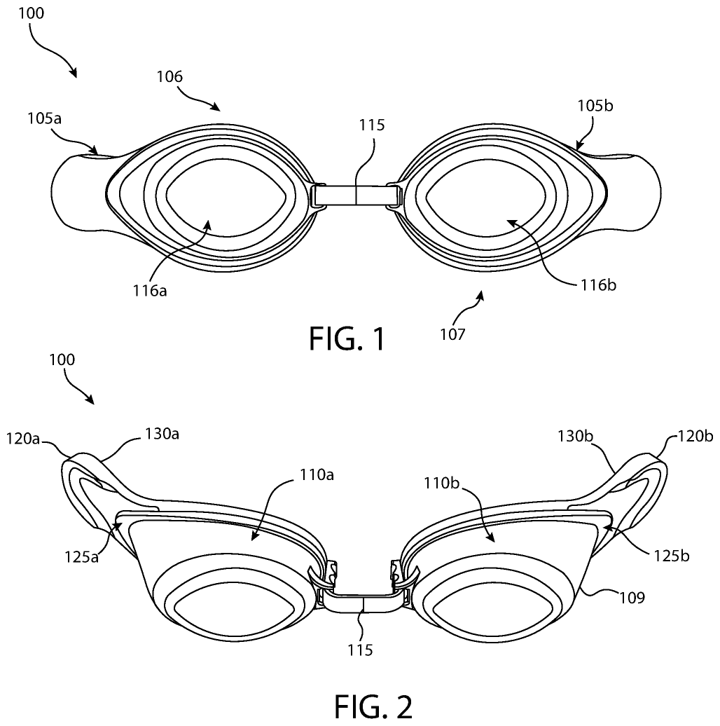

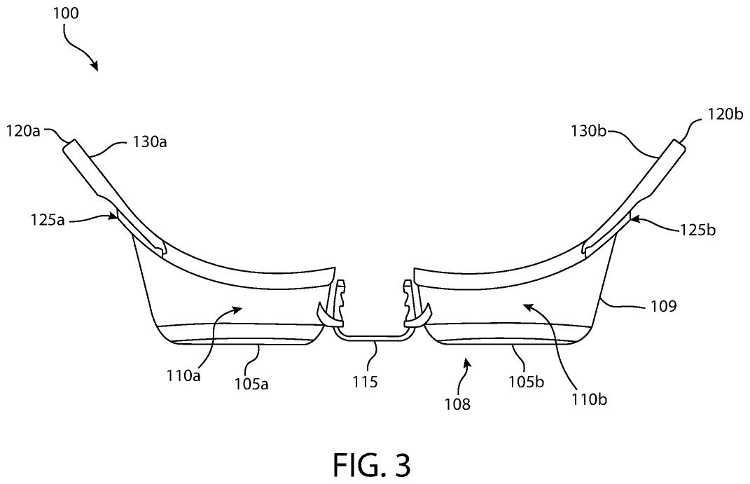

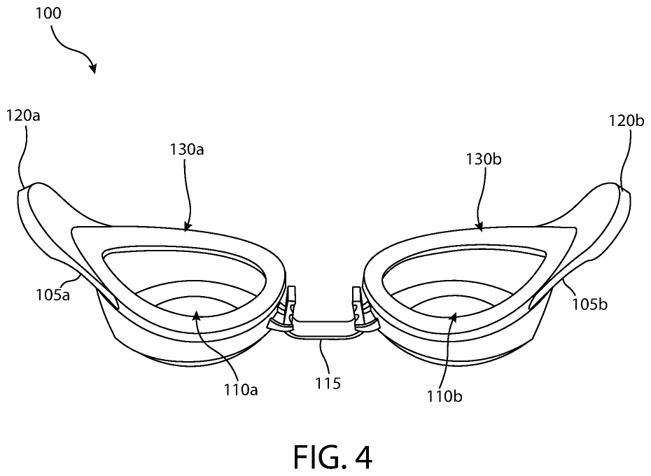

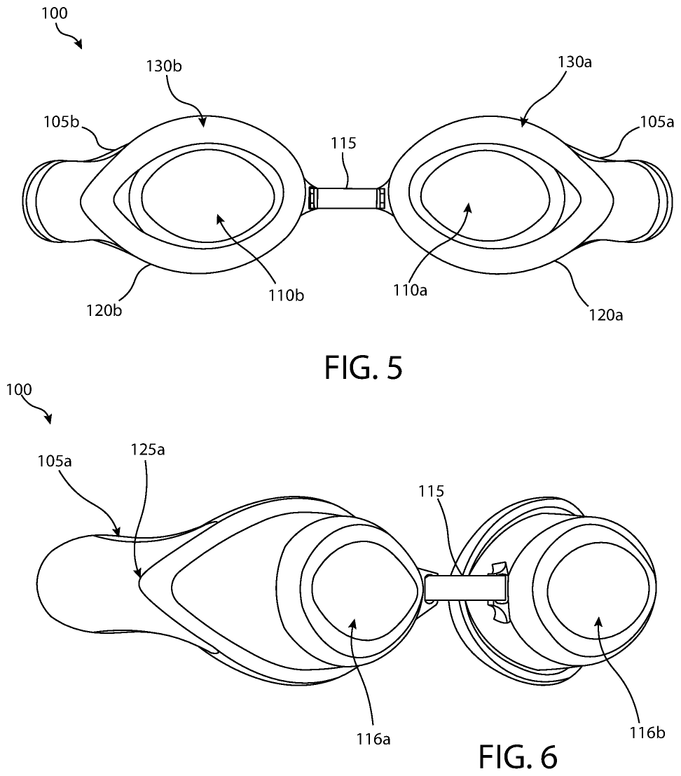

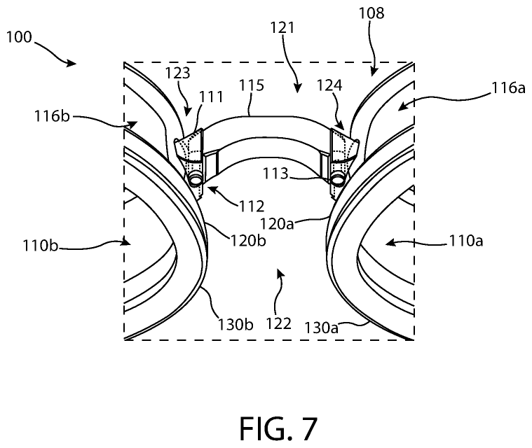

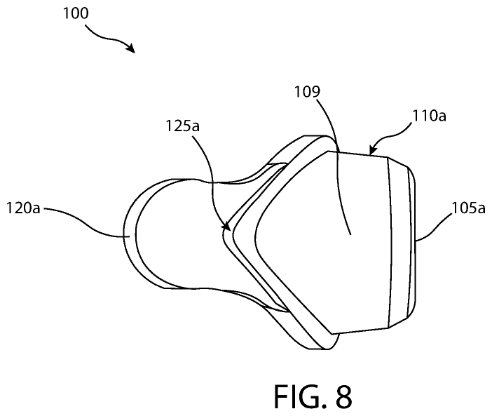

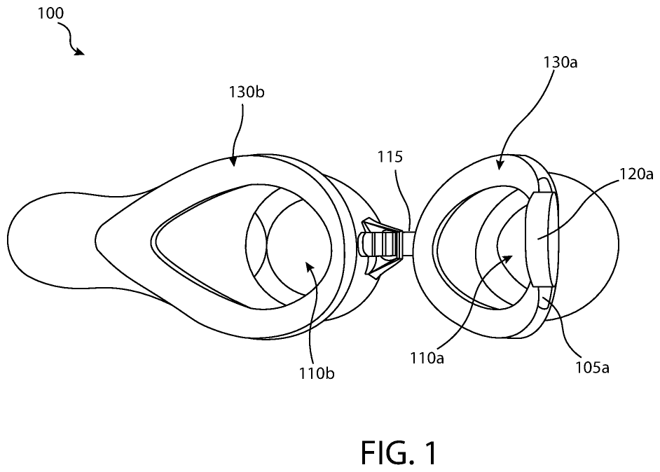

Referring now to the Figures, FIGS. 1-4 will be discussed together. FIGS. 1, 2, and 3 are front, rear and top views, respectively, of a system 100 for preventing damage to cosmetic eyelashes, according to an example embodiment of the present invention. FIG. 4 is a rear perspective view of a connector member connecting the eye members to each other, according to an example embodiment of the present invention. The system 100 includes a pair of eye members 105(a), 105(b), each having an interior volume 110(a), 110(b) that is configured to allow cosmetic lashes to move freely within the interior volume. In the present embodiment, each eye member has a substantially ovular shaped body comprising an upward facing side 106, a downward facing side 107, a front lens portion 108, an annular peripheral wall 109 extending rearward from a peripheral edge of the lens portion, and a flange III extending radially outward from the peripheral wall. Each flange includes a mounting hole 112 for snappingly engaging with a cylindrical joint 113 of a connector member 115, further discussed below. It should be appreciated that the eye members can have other shapes and dimensions, and such variations are within the spirit and scope of the claimed invention. The eye members can also be configured to be used with corrective or prescriptive lenses, polarized lenses, or nonprescription lenses, such as with clear accessory lenses to alter the appearance or color, or to provide protection from ultraviolet (IN) rays (UV-A and UV-B type). The eye members can be comprised of materials such as standard plastic, high-index plastic, polycarbonate, trivex, hard resin (CR-39), and crown glass.

The system further includes a connector member 115 for connecting the eye members 105(a), 105(b) to each other. The connector 115 is an elongated shaped body having flexible properties and is attached to each eye member 105(a), 105(b) such that the user's nose does not affect how each eye member removably attaches to the user's face when the user wears the system 100. In the present embodiment, the connector has a substantially C-shaped body comprising an upward facing side 121, a downward facing side 122, a first end 123, and a second end 124. A cylindrical joint 113 is located on the downward facing side of the first end and the second end. The cylindrical joints are configured to be inserted into the mounting holes 112 on the flange III, thereby connecting the eye members 105(a), 105(b) to each other. It should be appreciated that the connector can have other shapes and dimensions, and such variations are within the spirit and scope of the claimed invention. The connector can be made from any elastomeric thermoplastic material (plasticized PVC, thermoplastic rubber, polyurethane, etc.) or vulcanizable material (natural or synthetic rubber, neoprene, EPDM, solid or liquid silicone rubber, etc.).

Each eye member 105(a), 105(b) has a transparent portion 116(a), 116(b) and a rim 120(a), 120(b) on a rear edge 125(a), 125(b) of each eye member 105(a), 105(b). In the present embodiment, the rim has a substantially curved body to follow the contours of the wearer's face at the points of contact with the face. It should be appreciated that the rim can have other shapes and dimensions, and such variations are within the spirit and scope of the claimed invention The rim 120(a), 120(b) of each eye member can be made from any elastomeric thermoplastic material (plasticized PVC, thermoplastic rubber, polyurethane, etc.) or vulcanizable material (natural or synthetic rubber, neoprene, EPDM, solid or liquid silicone rubber, etc.). The transparent portion may be comprised of transparent polymer or plastic type materials. In some embodiments, each eye member 105(a), 105(b) includes a prescription lens portion, such that the user does not have to wear glasses or contacts.

An adhesive 130(a), 130(b) is disposed on each rim 120(a), 120(b). The adhesive 130(a), 130(b) is configured to removably attach the system 100 to a user's face. In the present embodiment, the adhesive 130(a), 130(b) is water resistant such that the system remains on the user's face when worn by the user while bathing or other activities without using a strap to attach the system 100 to the user's face. By eliminating the need of a strap to attach the system to the user's face, the adhesive improves blood circulation, and reduces ocular pressure for the wearer. The adhesive used in any of the embodiments of the invention may include adhesive compositions known in the art or hereafter developed for use in connection with a medical or surgical procedure.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A cosmetic lash protection device comprising:
a pair of eye members, each eye member including a rim defining an interior volume configured to allow the cosmetic lashes to move freely therein; and
an adhesive disposed on each rim, wherein the adhesive is configured to removably attach the device to a user's face.

2. The cosmetic lash protection device of claim 1, wherein the adhesive is replaceable.

3. The cosmetic lash protection device of claim 2, wherein the adhesive is water resistant such that the eyelash protection device remains on the user's face when worn by the user while bathing or other activities without using a strap to attach the eyelash protection device to the user's face.

4. The cosmetic lash protection device of claim 1, further comprising a connector member connecting the eye members to each other.

5. The cosmetic lash protection device of claim 4, wherein the connector includes an elongated shaped body.

6. The cosmetic lash protection device of claim 4, wherein the connector includes a flexible configuration.

7. The cosmetic lash protection device of claim 4, wherein the connector is attached to each eye member such that the user's nose does not affect how the eye members removably attaches to the user's face when the user wears the eyelash protection device.

8. The cosmetic lash protection device of claim 4, further comprising an attachment mechanism configured to attach the connector member with the pair of eye members.

9. The cosmetic lash protection device of claim 8, wherein the attachment mechanism includes a flange extending from each of the eye members.

10. The cosmetic lash protection device of claim 9, wherein each flange includes a mounting hole.

11. The cosmetic lash protection device of claim 10, wherein the attachment mechanism includes a cylindrical joint disposed with the connector member and configured for engagement with the mounting hole.

12. The cosmetic lash protection device of claim 1, wherein each of the eye members include a transparent lens portion.

13. The cosmetic lash protection device of claim 1, wherein each of the eye members include a prescription lens portion.

14. A cosmetic lash protection device comprising:
a pair of eye members, each eye member including a rim and an interior volume configured to allow the cosmetic lashes to move freely therein;
a connector member;
an attachment mechanism configured for connecting the eye members with the connector member; and
an adhesive disposed on each rim, wherein the adhesive is configured to removably attach the device to a user's face.

15. The cosmetic lash protection device of claim 14, wherein the attachment mechanism includes a flange extending from each of the eye members.

16. The cosmetic lash protection device of claim 15, wherein each flange includes a mounting hole.

17. The cosmetic lash protection device of claim 16, wherein the attachment mechanism includes a cylindrical joint disposed with the connector member and configured for engagement with the mounting hole.

18. The cosmetic lash protection device of claim 14, wherein each of the eye members include a transparent lens portion.

19. The cosmetic lash protection device of claim 14, wherein each of the eye members include a prescription lens portion.

20. A cosmetic lash protection system comprising:
a user wearing a pair of cosmetic lashes;
a pair of eye members configured for placement over the eyes of the user, each eye member including a rim defining an interior volume configured to allow the cosmetic lashes to move freely therein;
a connector member connecting the eye members to each other; and
an adhesive disposed on each rim, wherein the adhesive is configured to removably attach the system to a user's face.
